(19) United States
(12) Patent Application Publication
(10) Pub. No.: US 2004/0005636 A1
(43) Pub. Date: Jan. 8, 2004

(54) Method for obtaining the binding affinities of a peptide library to a protein

(76) Inventors: Rodney Kiplin Guy, Concord, CA (US); Jamie Marie Rasmussen Moore, San Francisco, CA (US); Timothy Ross Geistlinger, San Francisco, CA (US)

Correspondence Address: PENNIE AND EDMONDS, 1155 AVENUE OF THE AMERICAS, NEW YORK, NY, 100362711

(21) Appl. No.: 10/414,583

(22) Filed: Apr. 15, 2003

Related U.S. Application Data
(60) Provisional application No. 60/372,952, filed on Apr. 15, 2002.

(51) Int. Cl. G01N 33/53; C07K 14/72

(52) U.S. Cl. 435/7.1; 530/350; 530/358

(57) ABSTRACT

The present invention provides a method for measuring the binding of a peptide library to a target protein, both in the presence and absence of a ligand, or other activation modifier. The peptide library is chosen from known binding partners of the target protein, or members of the family to which it belongs. The members of the peptide library include a conserved interaction motif that permits them to bind to the target protein or its family. Individual peptides from the peptide library and the target protein are contacted with one another and a binding affinity measured. The binding affinities across the library are treated as a “fingerprint”. The method is preferably applied to a library of co-regulatory peptides that bind to a nuclear hormone receptor. The present invention further comprises the peptide library, and a composition comprising a member of the peptide library in contact with the target protein.

This application claims priority to U.S. Provisional Application Serial No. 60/372,952, filed Apr. 15, 2002, which is incorporated herein by reference in its entirety.

### FIELD OF THE INVENTION

The present invention provides a method for measuring a pattern of binding affinities for a peptide library to a protein. More particularly, the present invention provides a method wherein the peptide library is selected from known or inferred interacting partners of the protein, and those in its family.

### BACKGROUND OF THE INVENTION

Cells contain proteins that elicit a biological response by binding various molecules including other proteins, hormones, drugs, etc. Certain proteins engage more than one molecule when giving rise to a biological response. Such proteins, then, may require activation by a particular molecule, such as a cofactor, in conjunction with binding another molecule such as a ligand. Likewise, the activation may take the form of a post-translational modification such as phosphorylation. Alternatively, the activation may take the form of a structural rearrangement that occurs upon binding the ligand so that the binding site only recognizes the cofactor after the structural rearrangement has taken place. An example of a class of proteins that behaves in this manner is the family of nuclear hormone receptors. The drug discovery process for proteins that utilize an activator is therefore complicated by the need to understand not only the ligand-binding event, but the interaction with the activator.

The drug discovery process today has been profoundly affected by the development of library based technologies. Libraries, particularly peptide libraries and combinatorial libraries offer the possibility of screening many hundreds of compounds against a particular protein, in the quest for one or more highly active lead molecules that may provide a seed for a drug development project.

Although promising, library technologies suffer from problems of inefficiency. For example, it may be necessary to synthesize many thousands—possibly millions—of molecules in order to find a single highly active lead. Library technologies also suffer from the drawback that they may not add constructively to understanding of the behavior of the target. Not only is it difficult to deconvolute much of the library data, but most existing library technologies do not focus on sample purity. Accordingly, whatever data is obtained, is not of high quality. Thus making sense of binding data for a large number of randomly generated molecules rarely leads to an improved understanding of how the target behaves, or how the lead molecule may behave under physiological conditions. Furthermore, the fact that most of the randomly generated molecules have no natural counterpart and so are not able to exploit the types of interactions that actually occur means that, often, the most valuable information is not exploited to its fullest extent.

Proteins that utilize an activator, or activating mechanism, in conjunction with a binding event with a ligand, have been the focus of some library-based technologies. For example, previous methods for measuring the binding affinity of co-regulatory peptides to nuclear receptors include mammalian two-hybrid assays, in vitro pull down studies, and fluorescence polarization with single peptide probes (see, e.g., Lustig et al., International Publication WO 99/25635). However, none of the existing methods allows for the measurement of the binding affinity of a number of co-regulatory peptides for a nuclear receptor in a multiplex fashion. Such a limitation has hindered the development of high throughput assays for detecting and characterizing high affinity co-regulatory peptides.

Furthermore, although multiplex assays may be convenient for gathering large amounts of data, they typically only identify tight binders. It would be desirable to develop a method wherein a range of low and high affinity binding constants was determined, thereby producing a fingerprint based on a variegated selection of binding data.

Bramlett et al., Molecular Endocrinology, 15(6): 909-922, (2001), incorporated herein by reference, describe a time-resolved fluorescence-based multiplexed measurement of binding of 10 co-regulatory peptides containing a nuclear-receptor box (NR-box) motif to estrogen receptor subtypes from the family of nuclear receptors. This method, however, involves plate washing, and does not result in an accurate binding affinity. The method also only considers a small number of related peptides which were known to bind to the target in question.

Library-based methods represent one extreme of a potential continuum of approaches to studying drug interactions. At the other extreme of such a continuum is the study of individual library members. However, obtaining detailed understanding of a particular molecule and its physiological behavior requires more focused studies that are both intensive and time consuming because they focus on single species at a time.

It has been recognized, however, that utilizing an array of information for a single molecule may prove useful in a comparative context. Arrays of information have included various physicochemical properties, both measured and calculated, as well as binding data itself across a range of targets. Such methods look upon an array of data for a single molecule, that can also be obtained reproducibly for other molecules, as a “fingerprint”, i.e., a representation of the molecule, defined by its own behavior, that is somehow unique.

Thus, what is needed is a method that simultaneously measures the binding affinity of multiple peptides to a protein, but in such a manner that useful information at high accuracy is obtained. Such information can then be used predictively.

### SUMMARY OF THE INVENTION

The present invention provides a method for measuring a pattern of binding affinities for a peptide library to a target protein that is a member of a family of proteins, comprising: contacting each peptide in the peptide library with the target protein, wherein each peptide in the peptide library corresponds to a fragment of an interacting partner of the protein, or another protein in the same family, wherein the fragment contains an interaction motif between the interacting partner and the protein; and measuring the relative binding affinity of each peptide of the peptide library with the target protein, thereby producing a pattern of binding affinities for the peptide library to the target protein.

The method of the present invention further comprises a method for measuring a pattern of differential binding affinities for a peptide library to a target protein, wherein the differential binding affinity is obtained by further measuring the relative binding affinity of each peptide of the peptide library with the target protein when a ligand is present, and wherein the differential binding affinity for each peptide comprises a difference between the binding affinity with the ligand present and the binding affinity with the ligand absent. It is similarly possible to use the methods of the present invention to obtain a pattern of binding for the peptide library in the presence of one ligand with respect to a target protein, and to compare it to a pattern of binding obtained in the presence of a second ligand.

The method of the present invention additionally comprises obtaining a differential binding affinity by: contacting each peptide in a negative peptide library with the target protein, wherein each peptide in the negative peptide library corresponds to a peptide in the peptide library, wherein the interaction motif is modified; and measuring the relative binding affinity of each peptide of the negative peptide library with the protein; and obtaining a difference between the binding affinity of a negative peptide and the binding affinity of the peptide that corresponds to it in the peptide library. According to the methods of the present invention, the modified interaction motif of a negative peptide is such that the binding of the negative peptide to the target protein by the binding interaction that uses the interaction motif is effectively abolished.

In another embodiment of the method of the present invention, a differential binding affinity is obtained by comparing the pattern of binding affinities of a peptide library for different protein targets, or different protein subtypes, for example when bound to a given ligand. This represents a way of building up more detailed information about the binding characteristics of a given ligand.

The methods of the present invention are broadly applicable to any system that uses a regulated protein interaction and that can be modeled with a protein-peptide interaction. A “regulated” protein interaction is one wherein a secondary interaction, such as a post-translational modification, may change the affinity.

The present invention also provides a peptide library for obtaining a pattern of binding affinities to a target protein that is a member of a family of proteins, wherein each peptide in the peptide library corresponds to a fragment of an interacting partner, wherein the fragment contains an interaction motif between the interacting partner and a protein in the family of proteins.

The present invention further provides a peptide library for obtaining a pattern of binding affinities to a target protein that is a member of a family of proteins, wherein each peptide in the peptide library corresponds to a fragment of an interacting partner, wherein the fragment contains an interaction motif between the interacting partner and a protein in the family of proteins, and wherein an amount of the target protein is additionally in contact with each member of the library.

The members of the peptide library of the present invention are preferably provided by finding from the literature known interacting partners for any protein in the target protein's family, and thence generating a mimic of a portion of each known interacting partner that binds the protein. The mimic is preferably an independently synthesizable, or isolatable, fragment of the interacting partner that contains a consensus interaction motif, and which, independently, retains sufficient structural attributes of the interacting partner that it can mimic the binding behavior of the binding partner with respect to the protein.

Preferably, at least one member of the peptide library corresponds to a binding partner of a protein other than the target protein, but in the same family as the target protein. Additionally, it is preferable that the peptide library of the present invention contains peptides that correspond to interacting partners that don't necessarily bind to the target protein, but have a known binding interaction with at least one family member from the family that includes the target protein.

In one embodiment, a detectable label is covalently attached to each peptide of the peptide library. Preferably, the detectable label is a fluorescent label. More preferably, the fluorescent label is fluorescein. In such embodiments, binding affinity is measured by fluorescence polarization.

Preferably the peptide library includes peptides which bind to the protein with differing affinities. Preferably, each peptide of the peptide library is between about 15 and about 30 amino acid residues. More preferably, each peptide of the peptide library is between about 15 and about 25 amino acid residues. Even more preferably, each peptide of the peptide library is between 20 and about 30 amino acid residues.

Preferably the peptide library contains from as few as about 10 peptides to as many as several hundred—for example, about 300, about 400, or about 500—peptides. Preferably the libraries of the present invention contain from about 80 to about 120 peptides. Even more preferably, the libraries of the present invention contain from about 50 to about 80 peptides. Still more preferably, the libraries of the present invention contain from about 120 to about 200 peptides. In another preferred embodiment, the libraries of the present invention contain from about 25 to about 50 peptides. In still another preferred embodiment, the libraries of the present invention contain from about 15 to about 30 peptides.

In particular, the present invention further provides a method for measuring the binding of a peptide library of co-regulatory peptides to a protein such as a nuclear hormone receptor. In an especially preferred embodiment, the nuclear hormone receptor is ERα/β, PR, AR, GR, MR, RARα/β/γ, TRα/β, VDR, EcR, RXRα/β/γ, PPARα/β/γ, LXRα/β, FXR, PXR/SXR, CAR, SF-1, LRH-1, DAX-1, SHP, TLX, PNR, NGF1-Bα/β/γ, RORα/β/γ, ERRα/β/γ, GCNF, TR2/4, HNF-4, COUP-TFα/β/γ, wherein the abbreviations are those commonly used in the art. Preferred nuclear hormone receptors for use with the present invention include thyroid hormone (particularly TRβ), estrogen receptor (particularly ERα), and orphan receptors.

Preferably, then for use in conjunction with a nuclear hormone receptor, the peptide library is a library of co-regulatory peptides. Co-regulatory peptides include, generally, co-activators, and co-repressors, and peptides that have both functions. Co-regulatory peptides which may be used in the current invention include, but are not limited to, SRC-1, SRC-2 and SRC-3, PBP/DRIP205/TRAP220, TRAP100, PRIP, PGC1, RIP140, p300/CBP, ARA70, ARA55, DAX-1, SHP, NCoR, SMRT. Preferred co-regulatory peptides include peptides from SRC-1, SRC-2, SRC-3, NCoR, and SMRT. Typically there are 3-4 peptides from each of SRC-1, SRC-2 and SRC-3, wherein the numbering indicates that there are multiple interaction domains for each SRC protein. Thus, the present invention allows for the optimization of high throughput screening for nuclear hormone receptors, identification of function selective nuclear receptor ligands, and the identification and characterization of optimum co-regulatory peptides for orphan receptors.

### BRIEF DESCRIPTION OF THE DRAWINGS

FIG. 1 illustrates the binding of 3 representative peptides (solid lines) from a library of SRC-1 peptides to the ligand binding domain of TRβ, in comparison to negative control peptides (dashed lines) wherein L₂ and L₃ have been replaced by alanines.

FIG. 2 illustrates the binding of 3 representative peptides (solid lines) from a library of SRC-2 peptides to the ligand binding domain of TRβ, in comparison to negative control peptides (dashed lines) wherein L₂ and L₃ have been replaced by alanines.

FIG. 3 illustrates the binding of 3 representative peptides (solid lines) from a library of SRC-3 peptides to the ligand binding domain of TRβ, in comparison to negative control peptides (dashed lines) wherein L₂ and L₃ have been replaced by alanines.

FIG. 4 illustrates a Scatchard plot that shows binding of SRC2-2 peptide to the ligand binding domain of TRβ. The Scatchard plot is best fit to a linear function indicating a single class of binding sites.

FIG. 5 illustrates a Hill plot that shows binding of SRC2-2 peptide to TRβ. The slope of the Hill plot is approximately one indication of a single class of binding sites.

FIG. 6 illustrates the binding of SRC2-2 peptide to the ligand binding domain of ERα in the presence of estradiol, diethylstilbestrol and genistein.

FIG. 7 illustrates the binding of a library of SRC peptides to the ligand binding domain of ERα in the presence of tamoxifen.

### DETAILED DESCRIPTION OF THE INVENTION

Reference will now be made to preferred embodiments of the present invention. While the invention will be described in conjunction with the preferred embodiments, it will be understood that it is not intended to limit the invention to those preferred embodiments. To the contrary, the invention is intended to include alternatives, modifications, and equivalents as may be included within the spirit and scope of the invention as defined by the appended claims.

Overview

The present invention provides a method for measuring a pattern of binding affinities for a peptide library to a protein. In parallel, each member of the peptide library is contacted with the protein, and the relative binding affinity of the members of the peptide library to the protein is measured, thereby producing a pattern of binding affinities. Preferably, when a member of the peptide library is contacted with the protein, a binding species such as a ligand is also present. A pattern of binding is useful because it may be correlated to physiological events.

In practicing the present invention, one of ordinary skill in the art will identify a target protein that is a member of a family of proteins, and an interaction mechanism between the target protein and a number of binding partners. The target protein is hereinafter referred to as the “protein” to the extent that such a reference is unambiguous. The target protein can potentially be any protein of interest, whose function is desired to be understood.

By “family” of proteins, it is meant that there is a set of gene products, and possibly some inferred gene products, that are defined by a certain level of homology at the encoded amino acid residue level. Typically, proteins in the same family perform similar functions or roles. Some members of a protein family are inferred members because genes with an appropriate level of sequence homology have been identified, though whether or not they are actually expressed is open to question. Inferred members of a protein family may sometimes be found to have functions that are different from other members of the family, though for the purposes of the present invention, it is preferable that they share the same functionality as other members of the family. For example, there are approximately 50 known members of the nuclear receptor family, of which about 20 have known functions, and 35 have been named. The remaining 15 members are inferred.

Preferably, it will be possible to identify a conserved motif that underlies the interaction between members of the family of the target protein, and the binding partners. For example, methods of genetics may be used to identify such a motif. An example of such a motif is “LxxLL” (SEQ ID NO: 1) for coregulators of the nuclear receptor family.

Preferably, as many binding partners of the target protein and other members of its family as possible will be located. More preferably, all known binding partners will be located. Binding partners may be found by surveying the literature, searching bioinformatics databases, and by using other techniques familiar to one of ordinary skill in the art, e.g., from genomics. Accordingly, the libraries of the present invention differ from those of the prior art that are based on randomly generated compounds, because the libraries of the present invention utilize known and inferred binding partners from the literature.

However, it is also possible to deduce library members by considering genes that are related by homology to genes that code for known binding partners. Where such an approach would generate very large libraries, other filtering methods can be applied to make the size of the library more practical. For example, any gene that encodes for a protein that contains the sequence “LxxLL” could be considered when generating a NR-protein binding library. However, genes that encode for, e.g., poly-leucine, could be filtered from the analysis.

When identifying a target and an interaction mechanism, it is preferable to understand an underlying applicable biophysical event: for example, the interaction between a nuclear hormone, a coregulator and a nuclear hormone receptor is a biophysical event that is consistent with the methods of the present invention.

It is preferable that the methods of the present invention are applied to proteins that require activation by a particular molecule, such as a cofactor, in conjunction with binding another molecule such as a ligand. Alternatively, the activation of such proteins may take the form of a structural rearrangement that occurs upon binding the ligand so that the binding site only recognizes the cofactor after the structural rearrangement has taken place. In general, however, the activation process may be any post-translational modification of the protein structure, such as a ligand-binding phosphorylation, a glycosylation, or a methylation.

In some embodiments of the present invention, most binding partners of a target protein are proteins themselves and may even be large proteins. In such situations it is not necessary to construct a library that consists of the protein binding partners. Preferably, by identifying the relevant interaction domain and a conserved interaction motif, it is possible to populate the library with peptides that correspond to the sections of protein sequence that immediately surround the interaction motif. Thus, such peptides may either be obtained through digestion or pruning of the binding partner, or by direct peptide synthesis of the peptide fragment in question.

The length of the peptide that is required in a particular embodiment of the present invention is determined by the nature of the binding interaction. Preferably, enough of the peptide sequence surrounding the interaction motif is provided to mimic the facets of protein structure that nature uses for selectivity. Preferably, the K_{d} for the interaction between the binding partner and the protein maps to the interaction between the peptide and the protein. The key is that the peptide that is used is able to interact with the protein in a manner that is very similar to the manner in which the binding partner does so. Of course, a peptide in solution may adopt many conformations, but for the purposes of the present invention, the peptide must be sufficiently unconstrained that it can adopt the conformation necessary to bind to the protein, thereby mimicking the binding event between the corresponding binding partner and the protein. It is thus particularly desirable to apply the methods of the present invention to situations where the binding event between the binding partner and the protein determines the induced fit structure of the bound pair, so that the precise conformation of the peptide fragment in solution is not critical to the binding event.

It is also consistent with the methods of the present invention that only a portion of the target protein needs to be used. In particular, it is sufficient to use a piece of the target protein that interacts with an activator such as a coregulator, as well as a ligand, as long as the 3-dimensional attributes of the structure have not been lost. For example, for a multi-domain protein, it may be possible to use just a single domain, or fewer domains than the total number.

An advantage of using a library of peptides generated in this manner is that it narrows the focus for subsequent discovery of ligands that may bind. The libraries of the present invention are superior to random libraries of peptides, or otherwise combinatorially generated molecules, because they are pre-loaded with knowledge that comes from known binding partners. It is not necessary to remove members from the library that are similar in structure, or binding behavior. When comparing binding patterns of a particular library obtained for different ligands, differences between library members may be revealed. Furthermore, the libraries of the present invention, because they contain peptides that correspond to binding partners of many different members from the same family as the target protein, are generally applicable to obtaining patterns of binding affinities against any proteins in the same family as the target protein. Thus, the same library may have general applicability to target proteins across a given family.

The libraries of the present invention may contain as many as several hundred—for example, about 300, about 400, or about 500—peptides, or as few as about 10 peptides. Preferably the libraries of the present invention contain from about 80 to about 120 peptides. Even more preferably, the libraries of the present invention contain from about 50 to about 80 peptides. In another preferred embodiment, the libraries of the present invention contain from about 25 to about 50 peptides. In still another preferred embodiment, the libraries of the present invention contain from about 15 to about 30 peptides. Libraries of such a manageable size may be screened routinely and also very precisely, thereby leading to data of high reliability. Thus, it is not intended that the individual members of the libraries of the present invention are screened together, as is often achieved in the multiplexed methods of the prior art. The methods of the present invention permit the individual library members to be screened separately from one another (but in parallel), in a manner that is either simultaneous as well as in batches over time.

It is especially preferred that the members of the libraries of the present invention are purified, so that the binding data that is obtained is of high quality.

Screening a library using the methods of the present invention leads to a “fingerprint” wherein the fingerprint comprises the binding affinities of the library members for the target protein. The fingerprint can be associated with a ligand that is optionally present during the binding of the library members to the protein. Thus, by screening the library in the presence of a second ligand, a second fingerprint can be obtained, and used for the purposes of comparison of the properties of two ligands. Such fingerprints are useful because they describe how a ligand may behave in the body. The library is populated with naturally occurring binding partners—or their mimics—for a particular protein. Thus a fingerprint represents information about how a ligand would interact with the protein in the presence of each of a large number of naturally occurring binding partners. Thus, two ligands with similar fingerprints can be expected to behave in the same way physiologically. Such information is useful for predicting, for example, side effects of a drug.

Thus, a fingerprint can lead to a prediction of how a biological system will react in response to a given molecule. For example, it could be used to screen selective estrogen response modulators.

Proteins

The present invention is applicable to any protein. In particular, the present invention works for nuclear receptors, nuclear hormone receptors, G-protein coupled receptors (GPCR's), and protein-interaction domains such as the PDZ and SH2 domains.

Proteins used in the current invention may be advantageously produced by recombinant DNA technology using techniques well known in the art for expressing genes. These methods include, for example, in vitro recombinant DNA techniques, synthetic techniques, and in vivo genetic recombination. See, for example, the techniques described in Sambrook et al., “Molecular Cloning,” Cold Spring Harbor Laboratory Press, Cold Spring Harbor, N.Y., Vols. 1-3, (1989), and periodic updates thereof, and Ausubel et al., eds., (1989), “Current Protocols in Molecular Biology,” Green Publishing Associates, Inc., and John Wiley & Sons, Inc., New York. DNA and RNA encoding any nuclear receptor hormone may be chemically synthesized using, for example, synthesizers. See, for example, the techniques described in “Oligonucleotide Synthesis”, (1984), Gait, M. J. ed., GIRL Press, Oxford.

A variety of host-expression vector systems may be utilized to express proteins for use with the methods of the present invention, as would be understood by one of ordinary skill in the art. The expression systems that may be used for purposes of the invention include selection systems that may be used in eukaryotic systems. Proteins can also be expressed in a prokaryotic cell using expression systems known to those of skill in the art. Expression systems useful for the practice of the current invention are described in U.S. Pat. Nos. 5,795,745; 5,714,346; 5,637,495; 5,496,713; 5,334,531; 4,634,677; 4,604,359; 4,601,980, all of which are incorporated herein by reference in their entirety.

Peptide Libraries

Peptide libraries used in the current invention may be synthesized using conventional methods of peptide synthesis well-known to the artisan of ordinary skill (e.g., solid phase synthesis using Boc (butyloxy carbonyl) protected amino acids and carbodiimide bond formation, solid phase synthesis using Fmoc (fluorenyl methyloxycarbonyl) protected amino acids and carbodiimide bond formation, solution phase synthesis, etc.). Additionally, methods describing parallel synthesis of peptides are also well-known to those of skill in the art and are readily applicable to the synthesis of the peptide libraries used in conjunction with the methods of the present invention. It is therefore contemplated that any possible method known in the art may be used to prepare the peptide libraries used in the current invention. A particularly preferred method is parallel solid phase synthesis using Fmoc protected amino acids with 2-(1H-benzotriazole-1-yl)-1,1,3,3-tetramethyluronium hexafluorophosphate (“HBTU”), and diethylisopropylamine as reagents to form the peptide bond. As is known to an artisan of ordinary skill, automated methods may be employed in the peptide synthesis procedures used in conjunction with the methods of the present invention.

It is especially preferred that the peptide library members are purified prior to measuring their binding affinities. In a preferred embodiment, about 1 mg, at about 95% purity or greater, is produced for each peptide in the library. It is even more preferred that each member of the library has a purity of 98% or greater. It is most preferred that each member of the library has a purity of greater than 99%.

Preferably, the peptides of the peptide library are between 50 amino acid residues and 15 amino acid residues, and more preferably between 15 amino acid residues and 30 amino acid residues. In one preferred embodiment, the peptides of the peptide library are between 15 amino acid residues and 25 amino acid residues. In another preferred embodiment, the peptides of the peptide library are between 20 amino acid residues and about 30 amino acid residues.

According to the methods of the present invention, the member of the peptide library and the target protein are preferably in a homogeneous mixture and are permitted to reach equilibrium. Accordingly, in parallel, each member of the peptide library is equilibriated with the target protein, and the relative binding affinity of the members of the peptide library to the target protein is measured. In one embodiment, each member of the peptide library is accompanied by a ligand that binds to the target protein.

It is to be understood that, in the context of the present invention, when an operation on “each” member of the peptide library is described, such as contacting each member with the target protein, it is preferable that every member of the library is treated in the same way. However, it is also consistent with the present invention that the operation in question is carried out with a subset of the library, or substantially all of the library wherein a small number of library members amounting to up to about 5%, or several such members, are omitted. Such a circumstance may prevail if it is found that a small number of library members is not sufficiently pure to produce results of desired accuracy, or that the small number of library members has not been produced in sufficient yield.

The methods of the present invention give rise to the libraries that are composed of compounds that give rise to a wide range of K_{d}, thereby providing a useful fingerprint. This is possible because the peptides in the library mimic binding partners for any one or more proteins in the same family as the target protein.

The mixture of peptide, target protein and, optionally, ligand, may also contain a variety of other components such as salts, buffers, protease inhibitors, detergents, etc. Preferably, the components of the mixture are in solution. The components may be added in any order and may be incubated at any temperature which facilitates binding of the peptides to the target protein. The binding measurement is preferably carried out in a plate, such as a 96-well plate, or a 384-well plate. In this respect, the plate is used as a vessel, particularly one that facilitates parallel processing, and it is preferred that the target protein is not attached to the plate. Binding affinities are typically measured by standard methods of measuring K_{d}.

The methods of measuring binding affinity in conjunction with the present invention preferably use an homogeneous, equilibrium, format that does not involve washing. Typically, a mixture of protein, ligand and peptide library member will reach equilibrium in about 5 minutes. The fact that all of the components are all in the same physical state, i.e., in solution, dissolved in a buffer, means that there are no effects arising from adsorption on a surface.

After incubation, the relative binding affinity of the peptides of the peptide library to the nuclear hormone receptor may be determined. A number of methods may be used to detect binding between members of the peptide library and receptor (e.g. ultracentrifugation, circular dichroism, quenching of fluorescence, etc.) which depend on the label attached to the peptides of the peptide library.

Preferably, the peptides of the peptide library include a detectable label, which may be covalently attached. The detectable label may be directly detected by radioactivity, luminescence, fluorescence optical density, electrical density, etc. or may be indirectly detected (e.g., epitope tag). The label may be directly detected (e.g., through optical density, electrical density, energy transfer, etc.) or indirectly detected (by use of antibody conjugates, etc.).

Preferably, the label is a fluorescent label, which provides differential fluorescence polarization depending on whether the peptide is bound to the receptor. A preferred example of a fluorescent label is fluorescein. Other fluorescent labels are well know in the art (e.g., dansyl, rhodamine, etc.) and are within the scope of the present invention. Generally, labels may be attached to the peptides of the peptide library by conventional methods known to the skilled artisan (e.g., directly attached, attached through a linker to the N-terminal amine, to the free sulfhydryl of cysteine, etc.). Preferably, the library members are purified after the label has been added.

Preferably, the peptide library is used in a manner so that individual peptide members are at a concentration of less than about 1 μM, preferably, less than about 100 nM, and most preferably less than about 10 nM. In one especially preferred embodiment, the library members are present at a concentration of about 10 nM.

Optionally, a ligand may be present in the mixture of peptide and target protein. Suitable ligands include any possible compound which is capable of binding to the target protein in the presence of a peptide from the peptide library. Preferably, the ligand is an organic compound. More preferably the ligand is an organic compound of molecular weight less than about 750 daltons, and most preferably less than about 500 daltons. Ligands can be obtained from natural sources, partial synthesis, total synthesis, or compound libraries. Preferably, the ligand increases the binding affinity of at least some of the members of the peptide library for the target protein.

Differential Binding Affinities

Advantageously, the present invention may also be used to optimize screening for novel ligands, identify function selective ligands, and characterize co-regulatory peptides for high-throughput screening for orphan receptors.

Discovery of novel ligands with novel pharmacology is always desirable even when the target protein has known ligands. Assays for novel ligands may be optimized using the method of the present invention. For example, the method of the present invention can identify peptides from the peptide library that exhibit altered affinity in the presence of the original ligand. One or more of this subset of peptides could be used to screen libraries of potential ligands to find novel ligands that provide differential affinity.

One of the difficulties in establishing a high throughput screening assay for orphan receptors is that a positive control for ligand binding does not exist. A post-translational modification, such as phosphorylation, known to activate the receptor may allow for selection of the best co-regulatory peptide from a peptide library. Alternatively, the screen may be performed using all potential peptide probes. Thus, the effects of putative ligands can be assessed post-assay to find those ligands that induce differential affinities with one or more peptides of the peptide library.

Patterns of Binding Affinities

There are a number of methods of numerically comparing fingerprints that would be understood by one of ordinary skill in the art. Typical methods include statistical methods such as hierarchical clustering, pairwise t-testing, k-nearest neighbors method, and Anova. Some methods can be found in, e.g., Duda, R. O., Hart, P. E., and Stork, D. G., Pattern Classification, 2^{nd} Ed., (John Wiley & Sons, Inc., 2001).

Nuclear Hormone Receptors

An exemplary protein family, to which to apply the methods of the present invention is the family of nuclear receptors. Accordingly, the present invention provides a method that measures the binding affinity of peptide libraries to nuclear receptors. In one aspect, the present invention provides a method for measuring the binding of a peptide library to a nuclear hormone receptor from the family of nuclear receptors.

Nuclear hormone receptors bind both a ligand and a cofactor, specifically a coregulatory peptide. In general, the binding of the ligand induces a conformational change that facilitates the binding of the coregulatory peptide. Some nuclear hormone receptors are capable of binding a cofactor in the absence of a ligand.

Accordingly, in conjunction with the methods of the present invention, a ligand may be bound to a nuclear hormone receptor, thereby inducing a conformational change in the receptor. The peptides in the peptide library are contacted with the ligand-bound receptor, and their binding affinities measured, thereby producing a fingerprint. The procedure can be repeated with a different ligand, thereby producing a different fingerprint.

The peptide libraries for use in conjunction with nuclear hormone receptors are preferably populated with peptides derived from proteins that contain the “LxxLL” motif, wherein L is leucine and X is any amino acid. This set includes proteins that have been reported to bind to at least one Nuclear Receptor (NR) under one “state” (i.e., ligand or posttranslational modification) and as such have been identified as cofactors, as well as peptides derived from proteins that have arisen from computational searches for proteins that contain the LxxLL motif, and other similar characteristics to cofactors but whose physiological role has yet to be elucidated. Additionally, it is possible to study the binding of cofactors to nuclear receptors under several states and therefore the K_{d} is shifted up or down, depending on the state of the nuclear receptor. The cofactors also vary from Nuclear Receptor to Nuclear Receptor.

Nuclear hormone receptors used in the current invention may be produced by techniques known in the art, such as recombinant DNA technology. Nuclear hormone receptors may be expressed by a variety of host-expression vector systems as discussed hereinabove. For example, nuclear receptor hormones can be expressed in a prokaryotic cell using expression systems known to those of skill in the art, as discussed hereinabove.

Accordingly, the present invention provides a method for predicting the binding of a coregulatory protein to a particular nuclear hormone receptor in response to a particular ligand. Natural nuclear hormone receptors possess discrete functional domains, including a ligand binding domain (LBD), see, e.g., Maglesdorf, et al., Cell, 83, 841, (1995). Nuclear hormone receptors for use with the methods of the present invention encompass full length receptors as well as fragments of receptors that include at least the ligand binding domain of the receptor. Accordingly, any nuclear hormone receptor fragment, which allows for differential binding of a co-regulatory peptide in the presence or absence of ligand may be used in the current invention.

Nuclear hormone receptors which may be used in the present invention include, but are not limited to, receptors commonly known by the following abbreviations: ERα, ERβ, PR, AR, GR, MR, RARα, RARβ, RARγ, TRα, TRβ, VDR, EcR, RXRα, RXRβ, RXRγ, PPARα, PPARβ, PPARγ, LXRα, LXRβ, FXR, PXR, SXR, CAR, SF-1, LRH-1, DAX-1, SHP, TLX, PNR, NGF1-Bα, NGF1-Bβ, NGF1-Bγ, RORα, RORβ, RORγ, ERRα, ERRβ, ERRγ, GCNF, TR2/4, HNF-4, COUP-TFα, COUP-TFβ and COUP-TFγ. It is to be understood that the methods of the present invention are also to be practiced with members of the nuclear hormone receptor family not listed herein. Preferably, the nuclear hormone receptor is a thyroid hormone receptor or an estrogen receptor. More preferably, the nuclear hormone receptor is TRβ, or ERα. In still another embodiment, the nuclear hormone receptor may be an orphan receptor. In one preferred embodiment, the nuclear hormone receptor is ERα and the ligand is estradiol, diethylstilbestrol, genistein or tamoxifen.

Herein, the commonly-used abbreviations for receptors in the nuclear hormone receptor family are as presented in Table 0, hereinbelow. Although the abbreviation TR has been used to designate both the thyroid hormone receptor, and the testicular receptor, for the purposes of the instant application, the abbreviation TR will be taken to mean the thyroid receptor (or one of its subtypes), except where explicitly indicated to the contrary.

TABLE 0
Abbreviations for Receptors in the Nuclear Hormone Receptor family

| | |
|---|---|
| ER | Estrogen Receptor |
| PR | Progesterone Receptor |
| AR | Androgen Receptor |
| GR | Glucocorticoid Receptor |
| MR | Mineralocorticoid Receptor |
| RAR | Retinoic Acid Receptor |
| TRα,β | Thyroid Receptor [See also, Testicular Receptor] |
| VDR | Vitamin D3 Receptor |
| EcR | Ecdysone Receptor |
| RXR | Retinoic Acid X Receptor |
| PPAR | Peroxisome Proliferator Activated Receptor |
| LXR | Liver X Receptor |
| FXR | Farnesoid X Receptor |
| PXR/SXR | Pregnane X Receptor/Steroid and Xenobiotic Receptor |
| CAR | Constitutive Adrostrane Receptor |
| SF-1 | Steroidogenic Factor 1 |
| DAX-1 | Dosage sensitive sex reversal-adrenal hypoplasia congenital |
| | critical region on the X chromosome, gene 1 |
| LRH-1 | Liver Receptor Homolog 1 |
| SHP | Small Heterodimer Partner |
| TLX | Tail-less Gene |
| PNR | Photoreceptor-Specific Nuclear Receptor |
| NGF1-B | Nerve Growth Factor |
| ROR | RAR related orphan receptor |
| ERR | Estrogen Related Receptor |
| GCNF | Germ Cell Nuclear Factor |
| TR2/4 | Testicular Receptor |
| | [Note that Thyroid Receptor is also labelled “TR”] |
| HNF-4 | Hepatocyte Nuclear Factor |
| COUP-TF | Chicken Ovalbumine Upstream Promoter, Transcription |
| | Factor. |

According to the methods of the present invention, in parallel, each member of the peptide library is equilibriated with the nuclear hormone receptor, and the relative binding affinity of the members of the peptide library to the nuclear hormone receptor is measured. In a preferred embodiment, a ligand for the nuclear hormone receptor is included with each member of the peptide library when it is contacted with the nuclear hormone receptor.

Preferably, when the target is a nuclear hormone receptor, the peptide library is a library of co-regulatory peptides. More preferably, the co-regulatory peptides include: SRC-1, SRC-2, or SRC-3 (wherein SRC is steroid receptor coactivator); PBP/DRIP205/TRAP220 (wherein PBP is PPAR binding protein, DRIP is VDR interacting protein, TRAP is TR activating protein, and as is understood, the various designations PBP/DRIP205/TRAP220 represent the same protein); TRAP100, PRIP (wherein PRIP is PPAR interacting protein); PGC1 (wherein PGC1 is PPARγ coactivator); RIP140 (wherein RIP is receptor interacting protein); p300/CBP (wherein p300 indicates a protein that is ˜300 kD, and CBP is CREBs binding protein); ARA70 or ARA55 (wherein ARA is androgen receptor activator); DAX-1 (see Table 0); SHP (wherein SHP is small heterodimer partner); NCoR (wherein NCoR is nuclear receptor corepressor); and SMRT (wherein SMRT is silencing mediator of RAR and TR). Even more preferably, the co-regulatory peptides are SRC peptides.

When the target is a nuclear hormone receptor, the peptides in the peptide library preferably contain the sub-sequence L₁X₁X₂L₂L₃ (SEQ ID NO: 2) wherein L₁ is leucine, L₂ is leucine, alanine, isoleucine, valine, or methionine, and L₃ is leucine, alanine, or isoleucine, and X₁ and X₂ are independently any amino acid. Preferably, L₂ and L₃ are leucine. More preferably, L₂ and L₃ are independently leucine, alanine or isoleucine. Most preferably, L₂ and L₃ are either leucine or alanine.

The L₁X₁X₂L₂L₃ region of the peptide sequence typically forms an amphipathic alpha helix. The L₁X₁X₂L₂L₃ sub-sequence may be obtained from natural co-regulatory protein motif sequences, derived from co-regulatory protein motif sequences, or consensus sequences of co-regulatory protein motif sequences (obtained, for example, by step-wise mutational analysis and/or from screens of partial or completely synthetic sequences). The L₁X₁X₂L₂L₃ sub-sequence is abbreviated hereinafter to “LxxLL”.

Regions terminal to both the amino and carboxy terminus of the LxxLL sub-sequences are important in determining binding selectivity to nuclear hormone receptors. Preferably, the LxxLL sub-sequence is adjacent to amino and carboxy terminal regions that contain at least several amino acid residues (i.e., the LxxLL sub-sequence is not located close to either end of the peptide). Peptides which are shorter than about 15 amino acid residues are generally too short to fully model selectivity for binding to nuclear hormone receptors, i.e., the LxxLL sub-sequence is located too close to an end of such peptides.

Preferably, the peptides of the peptide library for use in conjunction with nuclear hormone receptors are between 15 amino acid residues and 50 amino acid residues, and more preferably between 15 amino acid residues and 30 amino acid residues. In one preferred embodiment, the peptides of the peptide library are between 15 amino acid residues and 25 amino acid residues. In another preferred embodiment, the peptides of the peptide library are between 20 amino acid residues and about 30 amino acid residues. In one preferred embodiment, the first or the last amino acid residue of a peptide in the peptide library is cysteine.

Optionally, a ligand may be present in the mixture of peptide library and nuclear hormone receptor. Suitable ligands include any possible compound which is capable of binding to the nuclear hormone receptor in the presence of a co-regulatory peptide from the peptide library. Many ligands for nuclear hormone receptors are already known and are within the scope of the present invention. In some situations, the ligand may increase the binding affinity of some of the peptides of the peptide library for the nuclear hormone receptor. Preferred ligands for the ERα receptor include estradiol, diethylstilbestrol, genistein or tamoxifen.

In a preferred embodiment, a fluorescent label is attached to each peptide in the peptide library used in conjunction with a nuclear hormone receptor, and fluorescence polarization is used to measure the relative binding affinities of the peptides of the peptide library to the nuclear hormone receptor.

The current invention may be used to facilitate screening for novel nuclear hormone receptor ligands, identify function selective nuclear receptor ligands, and characterize co-regulatory peptides for high-throughput screening for orphan receptors.

The method of the present invention can be used to identify co-regulatory peptides that exhibit altered affinity in the presence of the original ligand. One or more of this subset of peptides could be used to screen libraries of potential ligands to find novel ligands that provide differential affinity.

Ligands for nuclear hormone receptors may also be distinguished by effect on receptor conformation. By way of illustration, two ligands of the same affinity may cause the formation of different co-regulatory binding surfaces in the nuclear receptor ligand binding domain, and thus cause differential recruitment of co-regulatory proteins or peptides, which could then possess different pharmacological effects.

For example, selective estrogen receptor modulators (SERMs) cause the formation of different ER LBD conformations and have tissue specific profiles in respect of agonism and antagonism of estrogen signaling that correspond to these conformations. In fact, these ligands may regulate nuclear receptor signaling in one cell type but not another, or at one gene promoter and not another. The current invention may allow for rapid comparison of the binding of co-regulatory peptides to multiple ligand receptor pairs.

Furthermore, since the cofactor proteins used with nuclear hormone receptors are relatively large proteins, they contain multiple “LxxLL” motifs, and therefore multiple interaction domains. Therefore, the methods of the present invention can be used to identify which domain of the cofactor binds to the nuclear receptor under the different states.

### EXAMPLES

The invention is further defined by reference to the following examples, which describe the fluorescence polarization assay, the combinatorial synthesis of an SRC peptide library, and the measurement of the binding of this library to TRβ and ERα.

### Example 1

### Synthesis of Peptide Library

Coregulator peptides consisting of 20 amino acids with the general motif of CXXXXXXXLXX[L/A][L/A]XXXXXXX (SEQ ID NO: 3) were constructed, where C is cysteine, L is leucine, A is alanine, and X is any amino acid. The sequences of all of the coregulator peptides were obtained from human isoforms of proteins known to interact, biochemically or genetically, with one or more nuclear receptors.

The peptides were synthesized in parallel using standard fluorenyl methoxycarbonyl (Fmoc) chemistry in 48-well synthesis blocks (FlexChem System, Robbins). Preloaded Wang (Novagen) resin was deprotected with 20% piperidine in dimethylformamide. The next amino acid was then coupled using HBTU (2.38 equiv. wt.), Fmoc-protected amino acid (2.5 equiv. wt.), and diisopropylethylamine (5 equiv. wt.) in anhydrous dimethylformamide. Coupling efficiency was monitored by the Kaiser Test. Synthesis then proceeded through a cycle of deprotection and coupling steps until the peptides were completely synthesized.

The completed peptides were cleaved from the resin (81% TFA, 5% phenol, 5% thioanisole, 2.5% ethanedithiol, 3% water, 2% dimethylsulphide, 1.5% ammonium iodide) and crude product was dried down using a speedvac (GeneVac). Reversed-phase chromatography followed by mass spectrometry (electrospray ionization) was used to purify the peptides. The purified peptides were lyophilized. A thiol reactive fluorophore, 5-iodoacetamidofluorescein (Molecular Probes), was then coupled to the amino terminal cysteine following manufacturer protocol. Labeled peptide was isolated using reversed-phase chromatography and mass spectrometry.

Library members are shown in Table 1. Negative controls are shown in Table 2. Negative control peptides are peptides where L₂ and L₃ have been replaced with alanines. This mutation abolishes the interaction of co-regulatory peptide with the nuclear receptor, and therefore demonstrates that each co-regulatory peptide binds in a specific manner to the nuclear receptor via the LXXLL motif. The sequences in the 3^{rd} column of Tables 1 and 2 are presented so that the sequence “LxxLL” is aligned vertically.

TABLE 1

| NCBI Protein | | Sequence | |
|---|---|---|---|
| Accession Number | Peptide | \|LXXLL\| | |
| AAC50305 | SRC1-1 | CYSQTSHKLVQLLTTTAEQQ | SEQ ID NO:4 |
| | SRC1-2 | CLTARHKILHRLLQEGSPSD | SEQ ID NO:5 |
| | SRC1-3 | CESKDHQLLRYLLDKDEKDL | SEQ ID NO:6 |
| | SRC1-7 | CQAQQKSLLQQLLTE | SEQ ID NO:7 |
| Q15596 | SRC2-1 | CDSKGQTKLLQLLTTKSDQM | SEQ ID NO:8 |
| | SRC2-2 | CLKEKHKILHRLLQDSSSPV | SEQ ID NO:9 |
| | SRC2-3 | CKKKENALLRYLLDKDDTKD | SEQ ID NO:10 |
| Q9Y6Q9 | SRC3-1 | CESKGHKKLLQLLTCSSDDR | SEQ ID NO:11 |
| | SRC3-2 | CLQEKHRILHKLLQNGNSPA | SEQ ID NO:12 |
| | SRC3-3 | CKKENNALLRYLLDRDDPSD | SEQ ID NO:13 |
| AAF19083 | PGC-1 | CEAEEPSLLKKLLLAPANTQ | SEQ ID NO:14 |
| Q15648 | PBP-1 | CKVSQNPTLTSLLQITGNGG | SEQ ID NO:15 |
| | PBP-2 | CNTKNHPMLMNLLKDNPAQD | SEQ ID NO:16 |
| Q14686 | PRIP-1 | CVTLTSPLLVNLLQSDISAG | SEQ ID NO:17 |
| | PRIP-2 | CMREAPTSLSQLLDNSGAPN | SEQ ID NO:18 |
| Q75448 | TRAP100-1 | CRALLSALHWLLRCTAASA | SEQ ID NO:19 |
| | TRAP100-2 | CAFEFLLKLTPLLDKADQR | SEQ ID NO:20 |
| | TRAP100-3 | CHMLSGKSLDLLLAAAAATG | SEQ ID NO:21 |
| | TRAP100-4 | CDSTKVESLVALLNNSSEMK | SEQ ID NO:22 |
| | TRAP100-5 | CLVLLGHILPGLLTDSSKWH | SEQ ID NO:23 |
| | TRAP100-6 | CDDVQPSKLMRLLSSNEDDA | SEQ ID NO:24 |
| Q13772 | ARA70 | CLQQQAQQLYSLLGQFNCLT | SEQ ID NO:25 |
| NP_057011 | ARA55 | CLGTGLCELDRLLQELNATQ | SEQ ID NO:26 |
| Q92831 | p300 | CAASKHKQLSELLRSGSSPN | SEQ ID NO:27 |
| P48552 | RIP140-1 | CDSIVLTYLEGLLMHQAAGG | SEQ ID NO:28 |
| | RIP140-2 | CGKQDSTLLASLLQSFSSRL | SEQ ID NO:29 |
| | RIP140-3 | CYGVASSHLKTLLKKSKVKD | SEQ ID NO:30 |
| | RIP140-4 | CPSVACSQLALLLSSEAHLQ | SEQ ID NO:31 |
| | RIP140-5 | CQAANNSLLLHLLKSQTIPK | SEQ ID NO:32 |
| | RIP140-6 | CSHQKVTLLQLLLGHKNEEN | SEQ ID NO:33 |
| | RIP140-7 | CLLERRTVLQLLLGNPNKGK | SEQ ID NO:34 |
| | RIP140-8 | CSFSKNGLLSRLLRQNQDSY | SEQ ID NO:35 |
| | RIP140-9 | CESKSFNVLKQLLLSENCVR | SEQ ID NO:36 |
| AA032941 | NCoR-1 | CDPASNLGLEDIIRKALMGS | SEQ ID NO:37 |
| | NCoR-2 | CLITLADHICQIITQDFARN | SEQ ID NO:38 |
| | NCoR-3 | CTITAANFIDVTITRQIASS | SEQ ID NO:39 |
| Q9Y618 | SMRT-1 | CHASTNMGLEAIIRKALMGK | SEQ ID NO:40 |
| | SMRT-2 | CVVTLAQHTSEVTTQDYTRH | SEQ ID NO:41 |
| P51843 | DAX1-1 | CHQWQGSILYNMLMSAKQTR | SEQ ID NO:42 |
| | DAX1-2 | CHPRQGSILYSMLTSAKQTY | SEQ ID NO:43 |
| | DAX1-3 | CHPRQGSILYSLLTSSKQTH | SEQ ID NO:44 |
| Q15466 | SHP | CAASRPATLYALLSSSLKAV | SEQ ID NO:45 |

TABLE 2

| NCBI Protein | | Sequence | |
|---|---|---|---|
| Accession Number | Peptide | \|LXXAA\| | |
| AAC50305 | SRC1-1 | CYSQTSHKLVQAATTTAEQQ | SEQ ID NO:46 |
| | SRC1-2 | CLTARHKILHRAAQEGSPSD | SEQ ID NO:47 |
| | SRC1-3 | CESKDHQLLRYAADKDEKDL | SEQ ID NO:48 |
| | SRC1-7 | CQAQQKSLLQQAATE | SEQ ID NO:49 |
| Q15596 | SRC2-1 | CDSKGQTKLLQAATTKSDQM | SEQ ID NO:50 |
| | SRC2-2 | CLKEKHKILHPAAQDSSSPV | SEQ ID NO:51 |
| | SRC2-3 | CKKKENALLRYAADKDDTKD | SEQ ID NO:52 |
| Q9Y6Q9 | SRC3-1 | CESKGHKKLLQAATCSSDDR | SEQ ID NO:53 |
| | SRC3-2 | CLQEKHRILHKAAQNGNSPA | SEQ ID NO:54 |
| | SRC3-3 | CKKENNALLRYAADRDDPSD | SEQ ID NO:55 |
| AAF19083 | PGC-1 | CEAEEPSLLKKAALAPANTQ | SEQ ID NO:56 |
| Q15648 | PBP-1 | CKVSQNPILTSAAQITGNGG | SEQ ID NO:57 |
| | PBP-2 | CNTKNHPMLMNAAKDNPAQD | SEQ ID NO:58 |
| Q14686 | PRIP-1 | CVTLTSPLLVNAAQSDISAG | SEQ ID NO:59 |
| | PRTP-2 | CMREAPTSLSQAADNSGAPN | SEQ ID NO:60 |
| Q75448 | TRAP100-1 | CRALLSALHWAARCTAASA | SEQ ID NO:61 |
| | TRAP100-2 | CAFEFLLKLTPAADKADQR | SEQ ID NO:62 |
| | TRAP100-3 | CHMLSGKSLDLAAAAAAATG | SEQ ID NO:63 |
| | TRAP100-4 | CDSTKVESLVAAANNSSEMK | SEQ ID NO:64 |
| | TRAP100-5 | CLVLLGHILPGAATDSSKWH | SEQ ID NO:65 |
| | TRAP100-6 | CDDVQPSKLMRAASSNEDDA | SEQ ID NO:66 |
| Q13772 | ARA70 | CLQQQAQQLYSAAGQFNCLT | SEQ ID NO:67 |
| NP_057011 | ARA55 | CLGTGLCELDRAAQELNATQ | SEQ ID NO:68 |
| Q92831 | p300 | CAASKHKQLSEAARSGSSPN | SEQ ID NO:69 |
| P48552 | RIP140-1 | CDSIVLTYLEGAAMHQAAGG | SEQ ID NO:70 |
| | RIP140-2 | CGKQDSTLLASAAQSFSSRL | SEQ ID NO:71 |
| | RIP140-3 | CYGVASSHLKTAAKKSKVKD | SEQ ID NO:72 |
| | RIP140-4 | CPSVACSQLALAASSEAHLQ | SEQ ID NO:73 |
| | RIP140-5 | CQAANNSLLLHAAKSQTIPK | SEQ ID NO:74 |
| | RIP140-6 | CSHQKVTLLQLAAGHKNEEN | SEQ ID NO:75 |
| | RIP140-7 | CLLERRTVLQLAAGNPNKGK | SEQ ID NO:76 |
| | RIP140-8 | CSFSKNGLLSPAARQNQDSY | SEQ ID NO:77 |
| | RIP140-9 | CESKSFNVLKQAALSENCVR | SEQ ID NO:78 |
| AA032941 | NCoR-1 | CDPASNLGLEDAARKALMGS | SEQ ID NO:79 |
| | NCoR-2 | CLTTLADHTCQAATQDFARN | SEQ ID NO:80 |
| | NCoR-3 | CTITAANFIDVAATRQIASS | SEQ ID NO:81 |
| Q9Y618 | SMRT-1 | CHASTNMGLEAAARKALMGK | SEQ ID NO:82 |
| | SMRT-2 | CVVTLAQHISEAATQDYTRH | SEQ ID NO:83 |
| P51843 | DAX1-1 | CHQWQGSTLYNAAMSAKQTR | SEQ ID NO:84 |
| | DAX1-2 | CHPRQGSILYSAATSAKQTY | SEQ ID NO:85 |
| | DAX1-3 | CHPRQGSILYSAATSSKQTH | SEQ ID NO:86 |
| Q15466 | SHP | CAASRPAILYAAASSSLKAV | SEQ ID NO:87 |

### Example 2

### Fluorescence Polarization Assay of ERα or TRβ

The peptide library synthesized in Example 1 was assayed with ERα or TRβ receptor. Library members were kept at a constant concentration of 10 nM. A ligand was optionally added to the nuclear hormone receptor.

The receptor ERα-LBD or TRβ-LBD, expressed using reported protocols (for TR see, e.g., Darimont et al., Genes Dev., 12(21):3343-56, (1998); and for ER, see, e.g., Shiau et al., Cell, (1998), 95(7):927-937), was used. The concentration of protein varied between 0.001-20 μM in the following way. In 96 well plates, hTRβ-LBD or hERα-LBD was serially diluted from 40 μM to 0.002 μM in binding buffer (50 mM Sodium Phosphate, 150 mM NaCl, pH 7.2, 1 mM DTT, 1 mM EDTA, 0.01% NP40, 10% glycerol), containing 200 μM ligand (T₃, for TRβ, or estradiol, diethylstilbestrol, genistein or tamoxifen for ERα).

Subsequently 10 μL of diluted protein was added to 10 μL of fluorescent coregulator peptide (20 nM) in 384-well plates yielding final protein concentrations of 10-0.001 μM and 10 nM fluorescent peptide concentration. The samples were allowed to equilibriate for 30 minutes. Binding was then measured using fluorescence polarization (excitation λ at 485 nm, emission λ at 530 nm) on an Analyst AD (available from Molecular Devices).

Pilot experiments demonstrated that the binding of the coregulatory peptide SRC2-2 to hTRβ-LBD was completely saturable and reached equilibrium within 10 minutes and the observed K_{d} agreed well with the reported value of 1 μM. One of the major potential perturbing phenomena in this assay is unexpected quenching or enhancement of fluorescence intensity. To control for this, both overall fluorescence intensity and fluorescence anisotropy for each sample were simultaneously monitored.

All experiments were carried out in quadruplicate, with each iteration containing the positive and negative controls, and 10-12 dose points. The data were then fit using Klotz plots to determine IC₅₀ values using nonlinear regression analysis that fits the data to a modification of the model of Heyduk and Lee. (Heyduk et al., Proc. Natl. Acad. Sci., (1990), 1774; Heyduk et al., Nature, (1993), 364, 6437; Heyduk et al., Methods Enzymol., 274, 492. Further analysis was conducted using Hill and Scatchard plots to confirm number and homogeneity of binding sites as discussed hereinbelow.

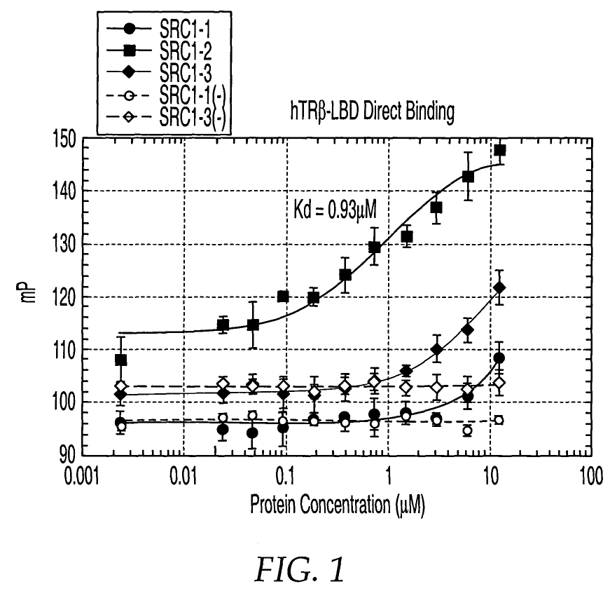

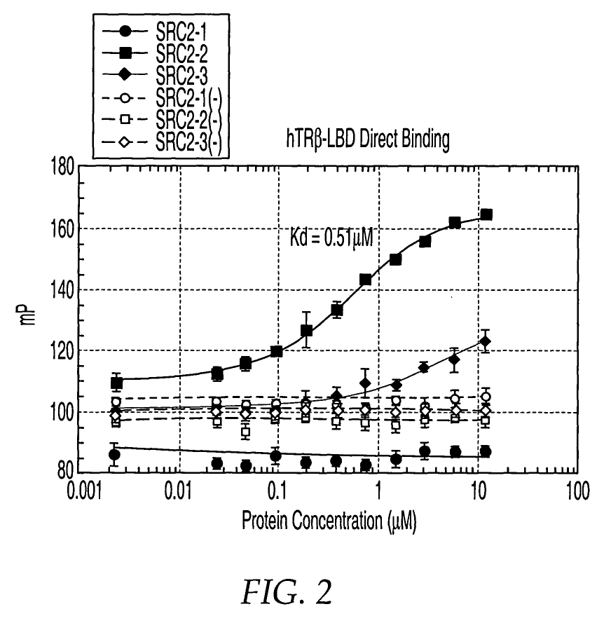

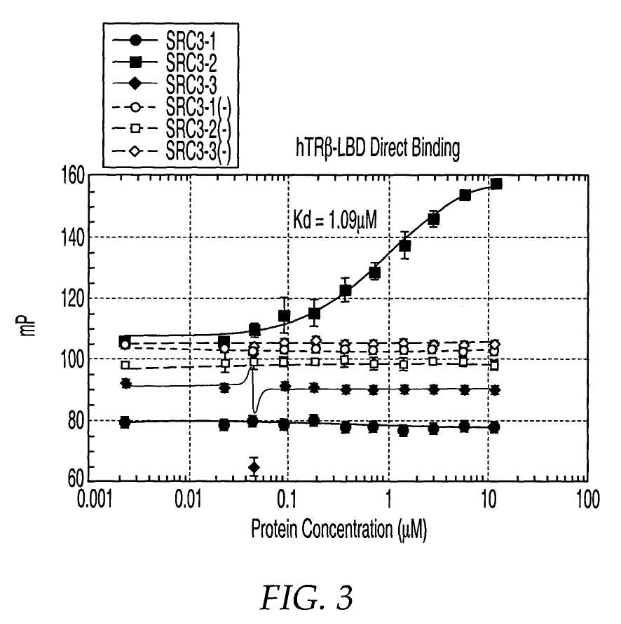

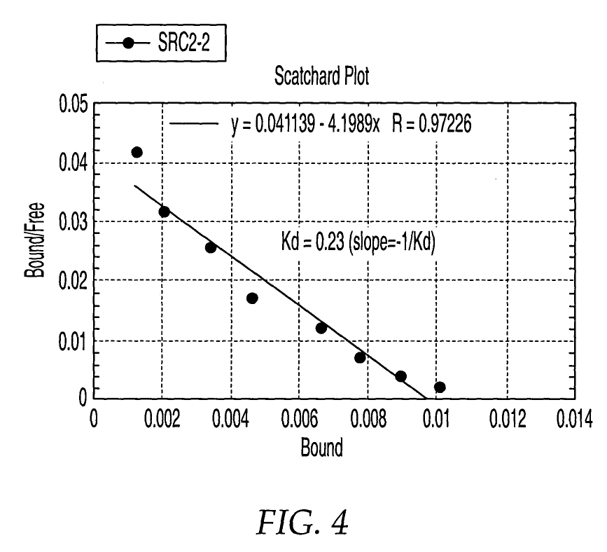

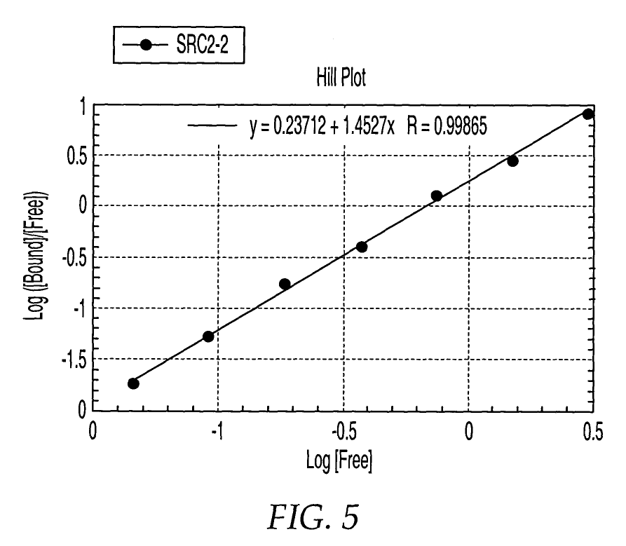

FIGS. 1 to 3 illustrate the results of three different measurements of the binding affinity of selected members of the SRC peptide library to TRβ-LBD. FIG. 4 illustrates a Scatchard plot, which provides the K_{d} for the SRC2-2 peptide. FIG. 5 illustrates a Hill plot, which provides the K_{d} for the SRC2-2 peptide. Both the Scatchard and Hill plot correlate with the direct binding plots illustrated in FIGS. 1-3. The Scatchard data were best fit to a linear function, which indicates a single class of binding sites. The slope of the Hill plot is 1, which also indicates a single class of binding sites. Measured dissociation constants for binding of the SRC peptide library to TRβ are illustrated in Table 3. Results are the mean of 2 independent assays run in quadruplicate; standard deviations are reported.

TABLE 3
Dissociation Constants for p160 peptides for TRβ

| p160 Peptide | Observed K_{d} | Published Results |
|---|---|---|
| SRC1-1 | >AL* | |
| SRC1-2 | 0.93 μM ± 0.15 | 2 > 3 > 1 |
| | | (Northrop et al., Molec. Endocrin- |
| | | ology, (2000), 14(5):605) 2 + 3 > 1 + 2 |
| | | (McInerney et al., Genes & Dev., |
| | | (1998), 12:3357) |
| SRC1-3 | >AL | |
| SRC1-7 | >AL | |
| SRC2-1 | >AL | does not interact |
| SRC2-2 | 0.67 μM ± 0.22 | 2 > 3 K_{d} app = 0.8 μM |
| | | (Darimont et al., Genes & Dev., |
| | | (1998), 12:3343) |
| SRC2-3 | ≧3 μM | 2 > 3 K_{d} app = 3.2 μM |
| | | (Darimont et al., Genes & Dev., |
| | | (1998), 12:3343) |
| SRC3-1 | 0.87 μM ± 0.31 | |
| SRC3-2 | >AL | |
| SRC3-3 | >AL | |

*AL = assay limits

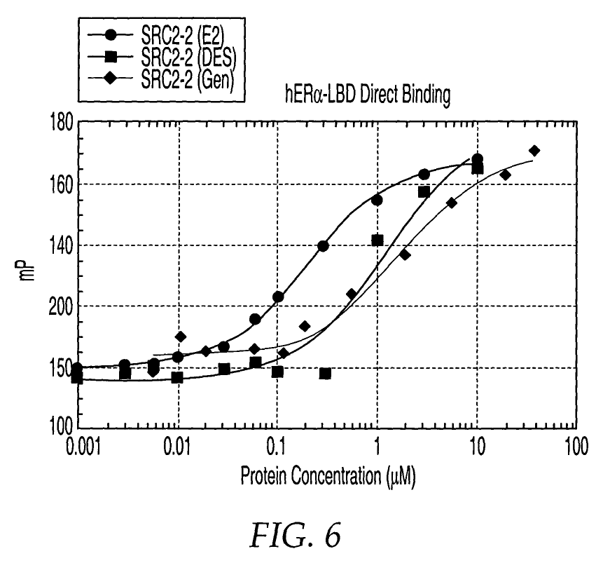

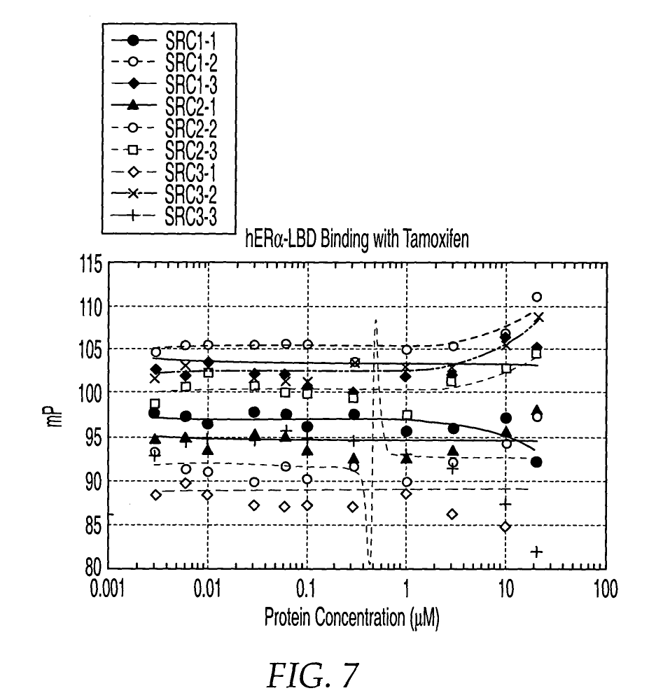

FIG. 6 illustrates direct binding of SRC2-2 to ERα-LBD in the presence of the ligands estradiol, diethylstilbestrol and genistein. SRC2-2 binds strongest to ERα in the presence of estradiol. FIG. 7 illustrates that the SRC library of Example 1 does not bind to ERα in the presence of tamoxifen. Measured dissociation constants for binding of members of the SRC peptide library to ERα, in the presence of 3 different ligands, are illustrated in Table 4 wherein the binding units are μM. Table 5 illustrates a comparison of binding constants (μM) for SRC peptides with the TRβ receptor in the presence of thyroid hormone T₃, and the ERα receptor in the presence of estradiol.

As can be seen by examining Tables 3, 4 and 5, SRC2-3, SRC2-2, SRC3-1, and SRC1-2 bind with approximately the same affinity to TRβand ERα, while the remaining peptides of the library have different affinities for the two receptors.

TABLE 4

| p160 Peptide | Estradiol | Genistein | Diethylstilbestrol |
|---|---|---|---|
| SRC1-1 | 1.206 | 5.000 | 0.853 |
| SRC1-2 | 0.231 | 0.298 | 0.302 |
| SRC1-3 | 2.540 | 2.550 | 1.463 |
| SRC2-1 | 0.927 | 2.601 | 0.896 |
| SRC2-2 | 0.222 | 0.569 | 0.277 |
| SRC2-3 | 1.417 | 2.000 | 0.786 |
| SRC3-1 | 0.160 | 0.250 | 1.000 |
| SRC3-2 | 0.363 | 1.030 | 0.415 |
| SRC3-3 | 100.000 | 100.000 | 100.000 |

TABLE 5

| p160 Peptide | TRβ/T₃ | ERα/Estradiol |
|---|---|---|
| SRC1-1 | 100.00 | 1.206 |
| SRC1-2 | 0.93 | 0.231 |
| SRC1-3 | 100.00 | 2.540 |
| SRC2-1 | 100.00 | 0.927 |
| SRC2-2 | 0.67 | 0.222 |
| SRC2-3 | 3.00 | 1.417 |
| SRC3-1 | 0.87 | 0.160 |
| SRC3-2 | 100.00 | 0.363 |
| SRC3-3 | 100.00 | 100.000 |

Finally, it should be noted that there are alternative ways of implementing the present invention. Accordingly, the present embodiments are to be considered as illustrative and not restrictive, and the invention is not to be limited to the details given herein, but may be modified within the scope and equivalents of the appended claims.

All references are incorporated herein by reference in their entirety.

What is claimed:

1. A method for measuring a pattern of binding affinities for a peptide library to a target protein that is a member of a family of proteins, comprising:
contacting each peptide in the peptide library with the target protein, wherein each peptide in the peptide library corresponds to a fragment of an interacting partner, wherein the fragment contains an interaction motif between the interacting partner and a protein in the family of proteins; and
measuring the relative binding affinity of each peptide of the peptide library with the target protein, thereby producing a pattern of binding affinities for the peptide library to the target protein.

2. The method of claim 1, further comprising purifying each peptide in the peptide library prior to contacting each peptide with the target protein.

3. The method of claim 1, wherein the peptide library comprises peptides that are representative of known proteinaceous cofactors, and are populated based on a consensus interaction motif.

4. The method of claim 1 wherein a detectable label is covalently attached to each peptide of the peptide library.

5. The method of claim 4, wherein the detectable label is a fluorescent label.

6. The method of claim 5, wherein the binding affinity is measured by fluorescence polarization.

7. The method of claim 1 wherein the target protein is a G-protein coupled receptor.

8. The method of claim 1 wherein the protein comprises an interaction domain that is PDZ or SH2.

9. The method of claim 1 wherein the target protein is a nuclear receptor.

10. The method of claim 9 wherein the nuclear receptor is a nuclear hormone receptor.

11. The method of claim 10, wherein the target protein comprises the ligand binding domain of the nuclear hormone receptor.

12. The method of claim 10, wherein the nuclear hormone receptor is selected from the group consisting of: ERα, ERβ, PR, AR, GR, MR, RARα, RARβ, RARγ, TRα, TRβ, VDR, EcR, RXRα, RXRβ, RXRγ, PPARα, PPARβ, PPARγ, LXRα, LXRβ, FXR, PXR, SXR, CAR, SF-1, LRH-1, DAX-1, SHP, TLX, PNR, NGF1-Bα, NGF1-Bβ, NGF1-Bγ, RORα, RORβ, RORγ, ERRα, ERRβ, ERRγ, GCNF, TR2/4, HNF-4, COUP-TFα, COUP-TFβ and COUP-TFγ.

13. The method of claim 10, wherein the nuclear hormone receptor is a thyroid hormone receptor or an estrogen receptor.

13. The method of claim 10, wherein the nuclear hormone receptor is TRβ or ERα.

14. The method of claim 10 wherein the nuclear hormone receptor is an orphan receptor.

15. The method of claim 10, wherein the peptide library is a library of co-regulatory peptides.

16. The method of claim 15, wherein the co-regulatory peptides are SRC-1, SRC-2, SRC-3, PBP/DRIP205/TRAP220, TRAP100, PRIP, PGC1, RIP140, p300/CBP, ARA70, ARA55, DAX-1, SHP, NCoR, SMRT.

17. The method of claim 16, wherein the co-regulatory peptide is SRC-1, SRC-2, SRC-3, NCoR, or SMRT.

18. The method of claim 15, wherein each peptide in the peptide library comprises the sequence L₁X₁X₂L₂L₃ wherein L₁ is leucine, L₂ is leucine, alanine, isoleucine, valine, or methionine, L₃ is leucine, alanine or isoleucine, and X₁ and X₂ are independently any amino acid.

19. The method of claim 18, wherein L₂ and L₃ are leucine.

20. The method of claim 18, wherein L₂ and L₃ are independently leucine, alanine, or isoleucine.

21. The method of claim 18, wherein a terminal amino acid residue of each peptide in the peptide library is cysteine.

22. The method of claim 1 wherein the target protein is activatable by contact with a peptide in the peptide library and by contact with a second species.

23. The method of claim 22 wherein the second species is a species capable of effecting a phosphorylation, glycosylation, or methylation of the target protein.

24. The method of claim 22 wherein the second species is a ligand which binds to the target protein.

25. The method of claim 1, wherein the contacting further comprises contacting a ligand for the target protein, with the target protein and the peptide from the peptide library.

26. The method of claim 25, wherein the ligand increases the binding affinity of at least one of the members of the peptide library for the target protein, relative to the binding affinity in absence of the ligand.

27. The method of claim 25, wherein the protein is ERα, and the ligand is estradiol, diethylstilbestrol, genistein or tamoxifen.

28. The method of claim 1 wherein the binding affinity is a differential binding affinity.

29. The method of claim 28 wherein the differential binding affinity is obtained by further measuring the relative binding affinity of each peptide of the peptide library with the target protein when a ligand is present, and wherein the differential binding affinity for each peptide comprises a difference between the binding affinity with the ligand present and the binding affinity with the ligand absent.

30. The method of claim 28 wherein the differential binding affinity is obtained by:
contacting each peptide in a negative peptide library with the target protein, wherein each peptide in the negative peptide library corresponds to a peptide in the peptide library, wherein the interaction motif is modified; and
measuring the relative binding affinity of each peptide of the negative peptide library with the protein; and
obtaining a difference between the binding affinity of a negative peptide and the binding affinity of the peptide that corresponds to it in the peptide library.

31. The method of claim 1, wherein each peptide of the peptide library has between about 15 and about 30 amino acid residues.

32. The method of claim 1, wherein each peptide of the peptide library has between about 15 and 25 amino acid residues.

33. The method of claim 1, wherein each peptide of the peptide library is between 20 and about 30 amino acid residues.

34. The method of claim 1, wherein the peptide library contains between about 10 and about 500 peptides.

35. The method of claim 1, wherein the peptide library contains between about 25 and about 50 peptides.

36. The method of claim 1, wherein the peptide library contains between about 15 and about 30 peptides.

37. The method of claim 1 wherein the contacting is carried out so that the member of the peptide library and the target protein are in a homogeneous mixture and are permitted to reach equilibrium.

38. A peptide library for obtaining a pattern of binding affinities to a target protein that is a member of a family of proteins, wherein each peptide in the peptide library corresponds to a fragment of an interacting partner, wherein the fragment contains an interaction motif between the interacting partner and a protein in the family of proteins.

39. The peptide library of claim 38, additionally comprising an amount of the target protein in contact with each member.


SEQUENCE LISTING (88 sequences)

SEQ ID NO: 1 - protein, 5 aa, Artificial
  Description of Artificial Sequence Motif
LXXLL

SEQ ID NO: 2 - protein, 5 aa, Artificial
  Description of Artificial Sequence Motif
LXXXX

SEQ ID NO: 3 - protein, 20 aa, Artificial
  Description of Artificial Sequence motif
CXXXXXXXLXXXXXXXXXXX

SEQ ID NO: 4 - protein, 20 aa, Homo sapiens
CYSQTSHKLVQLLTTTAEQQ

SEQ ID NO: 5 - protein, 20 aa, Homo sapiens
CLTARHKILHRLLQEGSPSD

SEQ ID NO: 6 - protein, 20 aa, Homo sapiens
CESKDHQLLRYLLDKDEKDL

SEQ ID NO: 7 - protein, 15 aa, Homo sapiens
CQAQQKSLLQQLLTE

SEQ ID NO: 8 - protein, 20 aa, Homo sapiens
CDSKGQTKLLQLLTTKSDQM

SEQ ID NO: 9 - protein, 20 aa, Homo sapiens
CLKEKHKILHRLLQDSSSPV

SEQ ID NO: 10 - protein, 20 aa, Homo sapiens
CKKKENALLRYLLDKDDTKD

SEQ ID NO: 11 - protein, 20 aa, Homo sapiens
CESKGHKKLLQLLTCSSDDR

SEQ ID NO: 12 - protein, 20 aa, Homo sapiens
CLQEKHRILHKLLQNGNSPA

SEQ ID NO: 13 - protein, 20 aa, Homo sapiens
CKKENNALLRYLLDRDDPSD

SEQ ID NO: 14 - protein, 20 aa, Homo sapiens
CEAEEPSLLKKLLLAPANTQ

SEQ ID NO: 15 - protein, 20 aa, Homo sapiens
CKVSQNPILTSLLQITGNGG

SEQ ID NO: 16 - protein, 20 aa, Homo sapiens
CNTKNHPMLMNLLKDNPAQD

SEQ ID NO: 17 - protein, 20 aa, Homo sapiens
CVTLTSPLLVNLLQSDISAG

SEQ ID NO: 18 - protein, 20 aa, Homo sapiens
CMREAPTSLSQLLDNSGAPN

SEQ ID NO: 19 - protein, 19 aa, Homo sapiens
CRALLSALHWLLRCTAASA

SEQ ID NO: 20 - protein, 19 aa, Homo sapiens
CAFEFLLKLTPLLDKADQR

SEQ ID NO: 21 - protein, 20 aa, Homo sapiens
CHMLSGKSLDLLLAAAAATG

SEQ ID NO: 22 - protein, 20 aa, Homo sapiens
CDSTKVESLVALLNNSSEMK

SEQ ID NO: 23 - protein, 20 aa, Homo sapiens
CLVLLGHILPGLLTDSSKWH

SEQ ID NO: 24 - protein, 20 aa, Homo sapiens
CDDVQPSKLMRLLSSNEDDA

SEQ ID NO: 25 - protein, 20 aa, Homo sapiens
CLQQQAQQLYSLLGQFNCLT

SEQ ID NO: 26 - protein, 20 aa, Homo sapiens
CLGTGLCELDRLLQELNATQ

SEQ ID NO: 27 - protein, 20 aa, Homo sapiens
CAASKHKQLSELLRSGSSPN

SEQ ID NO: 28 - protein, 20 aa, Homo sapiens
CDSIVLTYLEGLLMHQAAGG

SEQ ID NO: 29 - protein, 20 aa, Homo sapiens
CGKQDSTLLASLLQSFSSRL

SEQ ID NO: 30 - protein, 20 aa, Homo sapiens
CYGVASSHLKTLLKKSKVKD

SEQ ID NO: 31 - protein, 20 aa, Homo sapiens
CPSVACSQLALLLSSEAHLQ

SEQ ID NO: 32 - protein, 20 aa, Homo sapiens
CQAANNSLLLHLLKSQTIPK

SEQ ID NO: 33 - protein, 20 aa, Homo sapiens
CSHQKVTLLQLLLGHKNEEN

SEQ ID NO: 34 - protein, 20 aa, Homo sapiens
CLLERRTVLQLLLGNPNKGK

SEQ ID NO: 35 - protein, 20 aa, Homo sapiens
CSFSKNGLLSRLLRQNQDSY

SEQ ID NO: 36 - protein, 20 aa, Homo sapiens
CESKSFNVLKQLLLSENCVR

SEQ ID NO: 37 - protein, 20 aa, Homo sapiens
CDPASNLGLEDIIRKALMGS

SEQ ID NO: 38 - protein, 20 aa, Homo sapiens
CLITLADHICQIITQDFARN

SEQ ID NO: 39 - protein, 20 aa, Homo sapiens
CTITAANFIDVIITRQIASS

SEQ ID NO: 40 - protein, 20 aa, Homo sapiens
CHASTNMGLEAIIRKALMGK

SEQ ID NO: 41 - protein, 20 aa, Homo sapiens
CVVTLAQHISEVITQDYTRH

SEQ ID NO: 42 - protein, 20 aa, Homo sapiens
CHQWQGSILYNMLMSAKQTR

SEQ ID NO: 43 - protein, 20 aa, Homo sapiens
CHPRQGSILYSMLTSAKQTY

SEQ ID NO: 44 - protein, 20 aa, Homo sapiens
CHPRQGSILYSLLTSSKQTH

SEQ ID NO: 45 - protein, 20 aa, Homo sapiens
CAASRPAILYALLSSSLKAV

SEQ ID NO: 46 - protein, 20 aa, Homo sapiens
CYSQTSHKLVQAATTTAEQQ

SEQ ID NO: 47 - protein, 20 aa, Homo sapiens
CLTARHKILHRAAQEGSPSD

SEQ ID NO: 48 - protein, 20 aa, Homo sapiens
CESKDHQLLRYAADKDEKDL

SEQ ID NO: 49 - protein, 15 aa, Homo sapiens
CQAQQKSLLQQAATE

SEQ ID NO: 50 - protein, 20 aa, Homo sapiens
CDSKGQTKLLQAATTKSDQM

SEQ ID NO: 51 - protein, 20 aa, Homo sapiens
CLKEKHKILHRAAQDSSSPV

SEQ ID NO: 52 - protein, 20 aa, Homo sapiens
CKKKENALLRYAADKDDTKD

SEQ ID NO: 53 - protein, 20 aa, Homo sapiens
CESKGHKKLLQAATCSSDDR

SEQ ID NO: 54 - protein, 20 aa, Homo sapiens
CLQEKHRILHKAAQNGNSPA

SEQ ID NO: 55 - protein, 20 aa, Homo sapiens
CKKENNALLRYAADRDDPSD

SEQ ID NO: 56 - protein, 20 aa, Homo sapiens
CEAEEPSLLKKAALAPANTQ

SEQ ID NO: 57 - protein, 20 aa, Homo sapiens
CKVSQNPILTSAAQITGNGG

SEQ ID NO: 58 - protein, 20 aa, Homo sapiens
CNTKNHPMLMNAAKDNPAQD

SEQ ID NO: 59 - protein, 20 aa, Homo sapiens
CVTLTSPLLVNAAQSDISAG

SEQ ID NO: 60 - protein, 20 aa, Homo sapiens
CMREAPTSLSQAADNSGAPN

SEQ ID NO: 61 - protein, 19 aa, Homo sapiens
CRALLSALHWAARCTAASA

SEQ ID NO: 62 - protein, 19 aa, Homo sapiens
CAFEFLLKLTPAADKADQR

SEQ ID NO: 63 - protein, 20 aa, Homo sapiens
CHMLSGKSLDLAAAAAAATG

SEQ ID NO: 64 - protein, 20 aa, Homo sapiens
CDSTKVESLVAAANNSSEMK

SEQ ID NO: 65 - protein, 20 aa, Homo sapiens
CLVLLGHILPGAATDSSKWH

SEQ ID NO: 66 - protein, 20 aa, Homo sapiens
CDDVQPSKLMRAASSNEDDA

SEQ ID NO: 67 - protein, 20 aa, Homo sapiens
CLQQQAQQLYSAAGQFNCLT

SEQ ID NO: 68 - protein, 20 aa, Homo sapiens
CLGTGLCELDRAAQELNATQ

SEQ ID NO: 69 - protein, 20 aa, Homo sapiens
CAASKHKQLSEAARSGSSPN

SEQ ID NO: 70 - protein, 20 aa, Homo sapiens
CDSIVLTYLEGAAMHQAAGG

SEQ ID NO: 71 - protein, 20 aa, Homo sapiens
CGKQDSTLLASAAQSFSSRL

SEQ ID NO: 72 - protein, 20 aa, Homo sapiens
CYGVASSHLKTAAKKSKVKD

SEQ ID NO: 73 - protein, 20 aa, Homo sapiens
CPSVACSQLALAASSEAHLQ

SEQ ID NO: 74 - protein, 20 aa, Homo sapiens
CQAANNSLLLHAAKSQTIPK

SEQ ID NO: 75 - protein, 20 aa, Homo sapiens
CSHQKVTLLQLAAGHKNEEN

SEQ ID NO: 76 - protein, 20 aa, Homo sapiens
CLLERRTVLQLAAGNPNKGK

SEQ ID NO: 77 - protein, 20 aa, Homo sapiens
CSFSKNGLLSRAARQNQDSY

SEQ ID NO: 78 - protein, 20 aa, Homo sapiens
CESKSFNVLKQAALSENCVR

SEQ ID NO: 79 - protein, 20 aa, Homo sapiens
CDPASNLGLEDAARKALMGS

SEQ ID NO: 80 - protein, 20 aa, Homo sapiens
CLITLADHICQAATQDFARN

SEQ ID NO: 81 - protein, 20 aa, Homo sapiens
CTITAANFIDVAATRQIASS

SEQ ID NO: 82 - protein, 20 aa, Homo sapiens
CHASTNMGLEAAARKALMGK

SEQ ID NO: 83 - protein, 20 aa, Homo sapiens
CVVTLAQHISEAATQDYTRH

SEQ ID NO: 84 - protein, 20 aa, Homo sapiens
CHQWQGSILYNAAMSAKQTR

SEQ ID NO: 85 - protein, 20 aa, Homo sapiens
CHPRQGSILYSAATSAKQTY

SEQ ID NO: 86 - protein, 20 aa, Homo sapiens
CHPRQGSILYSAATSSKQTH

SEQ ID NO: 87 - protein, 20 aa, Homo sapiens
CAASRPAILYAAASSSLKAV

SEQ ID NO: 88 - protein, 5 aa, Artificial
  Description of Artificial Sequence Motif
LXXAA